(19) United States
(12) Patent Application Publication
(10) Pub. No.: US 2018/0317773 A1
(43) Pub. Date: Nov. 8, 2018

(54) IMAGING APPARATUS WITH TISSUE RETRIEVAL CHANNEL

(71) Applicant: NINEPOINT MEDICAL, INC., Bedford, MA (US)

(72) Inventors: Eman NAMATI, Concord, MA (US); Tsung-Han TSAI, Newton, MA (US); Peter G. STRICKLER, Tewksbury, MA (US)

(21) Appl. No.: 15/971,464

(22) Filed: May 4, 2018

Related U.S. Application Data
(60) Provisional application No. 62/502,622, filed on May 6, 2017.

(51) Int. Cl. A61B 5/00; A61B 10/02

(52) U.S. Cl. CPC A61B 5/0066; A61B 1/00165; A61B 10/02; A61B 5/0084

(57) ABSTRACT

Exemplary imaging apparatuses are described. Various embodiments of the imaging apparatuses may implement optical coherence tomography (OCT) and/or optical frequency domain imaging (OFDI) tissue imaging technologies, with the capability to perform tissue retrieval at the same time. Furthermore, the imaging apparatuses may include a rotatable imaging element to scan a bodily lumen, such as the bile duct. The imaging element may be housed within a cylindrical window. Still further, the imaging apparatuses may include an ancillary channel. The ancillary channel may provide access to the tissue in the bile duct, or other bodily lumen.

### CROSS-REFERENCE TO RELATED APPLICATIONS

This application claims the benefit of U.S. Provisional Application No. 62/502,622, filed May 6, 2017, which is incorporated herein by reference in its entirety.

### FIELD OF THE DISCLOSURE

The disclosure relates generally to the field of combined medical imaging and tissue management techniques, and more particularly to catheters for optical imaging and tissue retrieval.

### BACKGROUND OF THE DISCLOSURE

Histology is the current gold standard of disease diagnosis and typically requires retrieving tissue samples from the inner body. Examples of tissue retrieval techniques are incisional biopsy, needle aspiration biopsy, brush biopsy, and segmental resection.

Current histology based diagnostic yield in the inner body is limited due to the sampling error of tissue retrieval techniques which typically involve random or imprecise selection of small regions of interest.

Optical imaging of the inner body is an alternative method to assess anatomy and tissue structures, which can highlight the regions of interest and guide the tissue retrieval to further improve the diagnostic yield. Examples of optical imaging techniques are optical coherence tomography (OCT), fluoroscopy, and spectroscopy. Other exemplary methods include confocal, non-linear, and spectrally-encoded confocal microscopy (SECM).

Devices for optical imaging of the inner body include a distal imaging end functionally coupled to a proximal operating end. The imaging end is inserted into the body and is manipulated via the operating end accessible to an external operator.

One example device for optical imaging of the inner body is a fiber optic probe. Fiber optic probes may include an imager, at least one optical fiber, at least one illumination source, and an optical system. Fiber optic probes may also include other components which may be used to record the location of the probe inside the body, such as radiopaque markers and positional sensors.

Current devices for optical imaging of the inner body have a number of operational drawbacks for guiding tissue retrieval. For example, fiber-optic probes may not be used at the same time with the tissue retrieval tools due to limited space and may miss the targeted regions of interest.

It is with respect to these and other considerations that the present improvements are needed.

### SUMMARY OF THE DISCLOSURE

In view of the forgoing, exemplary imaging apparatuses are described. Various implementations of the imaging apparatuses may implement optical coherence tomography (OCT) and/or optical frequency domain imaging (OFDI) tissue imaging technologies, with the capability to perform tissue management at the same time. Furthermore, the imaging apparatuses may include a rotatable imaging element to scan a bodily lumen, such as the bile duct. The imaging element may be housed within a cylindrical window. Still further, the imaging apparatuses may include an ancillary channel. The ancillary channel may provide access to the tissue in the bile duct, or other bodily lumen.

### BRIEF DESCRIPTION OF THE DRAWINGS

By way of example, specific embodiments of the disclosed device will now be described, with reference to the accompanying drawings, in which:

FIGS. 1A and 1B illustrate a first exemplary imaging and tissue management apparatus.

FIGS. 2A and 2B illustrate a second exemplary imaging and tissue management apparatus.

FIGS. 3A and 3B illustrate a third exemplary imaging and tissue management apparatus.

FIGS. 4A and 4B illustrate a fourth exemplary imaging and tissue management apparatus.

### DETAILED DESCRIPTION

Various examples, implementations, and illustrative configurations are described herein. In some examples, the bile duct is used as an example bodily lumen. However, this is not intended to be limiting. Furthermore, the various depictions are not drawn to scale. Instead, they are drawn in a manner to facilitate understanding. Additionally, the various examples and illustrations can be combined with each other, even where not specifically so stated. Additionally, the described examples are not intended to limit the claims and present disclosure.

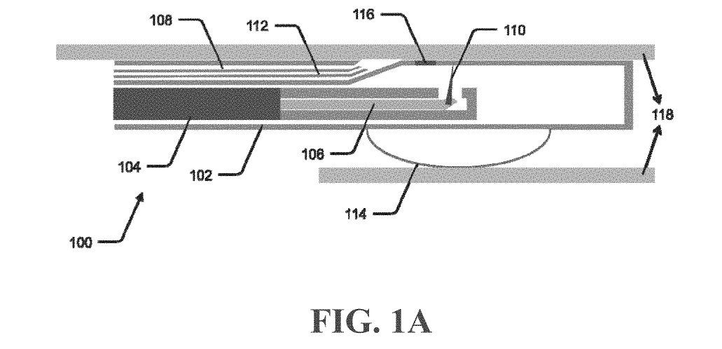

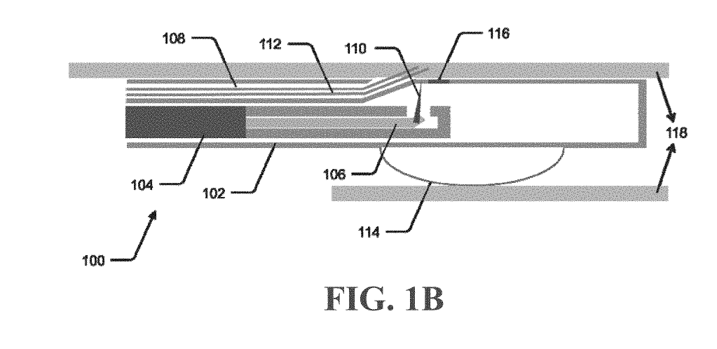

FIGS. 1A and 1B illustrate a first exemplary imaging and tissue management apparatus 100. The first exemplary imaging and tissue management apparatus 100 may implement optical coherence tomography (OCT) and/or optical frequency domain imaging (OFDI) tissue imaging methods and technologies. The first exemplary imaging and tissue management apparatus 100 may alternatively implement other scanning optical imaging modalities, such as fluorescence, spectroscopy, or the like. Furthermore, the imaging and tissue management apparatus 100 may implement other tissue imaging methods and technologies. In one implementation, the first exemplary imaging and tissue management apparatus 100 may include a system utilizing at least one of OCT or OFDI modalities. Specifically, the first exemplary imaging and tissue management apparatus 100 may be capable of detecting electromagnetic radiation, such as a back reflected light, from one or more portions associated with tissue 118. The detected electromagnetic radiation may be processed by the imaging and tissue management apparatus 100 to ascertain information, such as microstructures, associated with the tissue 118.

In one implementation, the first exemplary imaging and tissue management apparatus 100 may include a proximal system including optical fiber, tissue retrieval and treatment functionality, data processing and associated data storage, and the like. As illustrated in FIG. 1, the imaging and tissue management apparatus 100 includes a sheath 102. The sheath 102 may be generally associated with a catheter body. The sheath 102 may be made from a suitable transparent or translucent material such as fluorinated ethylene propylene (FEP), polytetrafluoroethylene (PTFE), polyphthalamide, polyimide, Nylon, or the like.

The sheath 102 may house an actuation translator 104. The actuation translator 104 enables rotation and translation of an imaging element 106 associated with actuation translator 104. The actuation translator 104 may actuate the proximal end of the imaging element 106, such as by torque coil, drive shaft, or the like. The actuation translator 104 may actuate the distal end of the imaging element 106, such as by motor, piezoelectric actuator, or the like.

The imaging and tissue management apparatus 100 may include one or more ancillary channels 108. The ancillary channel 108 may be alongside of the sheath 102. The ancillary channel 108 may be made from the same material as the sheath 102 such as fluorinated ethylene propylene (FEP), polytetrafluoroethylene (PTFE), polyphthalamide, polyimide, Nylon, or the like. The sheath 102 and the ancillary channel 108 may be made from a multi-lumen extrusion or a bundle of multiple tubing. The distal exit of the ancillary channel 108 may be at the location covered by the scanning range of an imaging beam 110. Multiple ancillary channels 108 may be included to enable greater coverage of the bodily lumen. In one implementation, the sheath 102 may be steerable via the proximal system to control the position or the orientation of the ancillary channel 108 to tissue 118 associated with the bodily lumen.

The ancillary channel 108 may house a tissue retrieval device 112. The tissue retrieval device 112 may be in the form of a biopsy forceps, an aspiration needle, or the like. The tissue retrieval device 112 will be in a retracted state in the ancillary channel 108 at the time the imaging and tissue management apparatus 100 is guided through the bodily lumen. The ancillary channel 108 enables deployment of the tissue retrieval device 112 at the targeted location. As an example, FIG. 1A illustrates the tissue retrieval device 112 in a retracted state while FIG. 1B illustrates the tissue retrieval device 112 in an extended or deployed state.

The imaging and tissue management apparatus 100 may include one or more balloons 114. The balloons 114 may be inflated via the sheath 102. The balloon 114 may be inflated with air, gas, liquid, or the like. The balloon 114 may be made from a suitable non-compliant, compliant, or semi-compliant material such as polyethylene or other polyolefins, polyurethane, flexible polyvinylchloride, Nylon, or the like. An exterior surface of the balloon 114 may be smooth or substantially smooth. Alternatively, the exterior surface of the balloon 114 may be textured with protuberances, or the like, to aid in anchoring the imaging and tissue management apparatus 100 to tissue associated with the bodily lumen.

The imaging element 106 may be coupled to a fiber optic line. The fiber optic line may be contained or housed within the sheath 102. The fiber optic line may be coupled to a portion of the imaging and tissue management apparatus 100 that enables OCT and/or OFDI methods and technologies. The imaging element 106 is functional for circumferential scanning by way of at least the rotation of actuation translator 104. Helical scanning can also be accomplished by simultaneous rotation and pull back by the actuation translator 104.

The imaging element 106 is capable of manipulating, directing, and/or focusing the imaging beam 110 on the tissue 118 during deployment of the tissue retrieval device 112. Light reflected from the tissue 118 may be processed by the imaging element 106 and conveyed to data processing systems associated with the imaging and tissue management apparatus 100 via the fiber optic line, or the like. The processed tissue information enables the guidance of the tissue retrieval process, such as by direct visualization of the tissue retrieval tool 112 or by the tissue 118 removed by the tissue retrieval tool 112. In one implementation, the reflected light is conveyed wirelessly to the data processing systems associated with the imaging and tissue management apparatus 100.

The sheath 102 may include one or more registration markers 116. The registration marker 116 may be associated with the sheath 102 in the portion covered by the scanning range of the imaging beam 110. The registration marker 116 may provide contrast for OCT/OFDI and at least one other imaging modality to enable positional registration, such as white light endoscopy, ultrasound, fluoroscopy, or the like. In one implementation, the registration marker 116 is made from radiopaque materials for visualization in fluoroscopy, such as barium sulfate, bismuth compounds, tungsten, or the like.

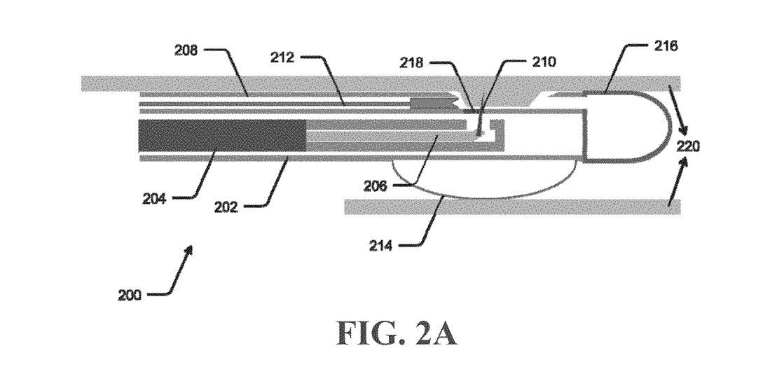

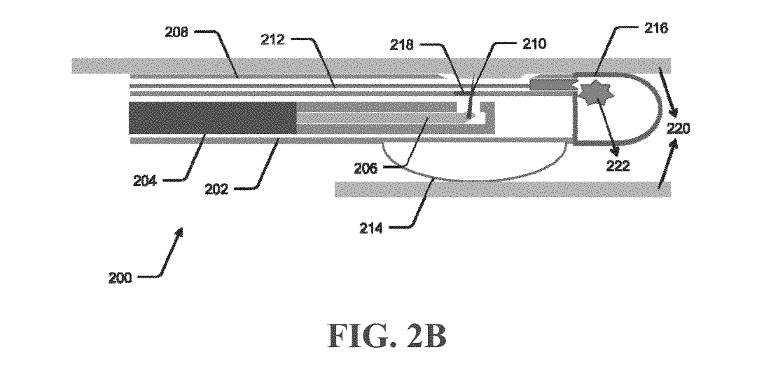

FIGS. 2A and 2B illustrate a second exemplary imaging and tissue management apparatus 200. The second exemplary imaging and tissue management apparatus 200 may implement optical coherence tomography (OCT) and/or optical frequency domain imaging (OFDI) tissue imaging methods and technologies. The second exemplary imaging and tissue management apparatus 200 may alternatively implement other scanning optical imaging modalities, such as fluorescence, spectroscopy, or the like. Furthermore, the imaging and tissue management apparatus 200 may implement other tissue imaging methods and technologies. In one implementation, the second exemplary imaging and tissue management apparatus 200 may include a system utilizing at least one of OCT or OFDI modalities. Specifically, the second exemplary imaging and tissue management apparatus 200 may be capable of detecting electromagnetic radiation, such as a back reflected light, from one or more portions associated with tissue 220. The detected electromagnetic radiation may be processed by the imaging and tissue management apparatus 200 to ascertain information, such as microstructures, associated with the tissue 220.

In one implementation, the second exemplary imaging and tissue management apparatus 200 may include a proximal system including optical fiber, tissue retrieval and treatment functionality, data processing and associated data storage, and the like. As illustrated in FIG. 2, the imaging and tissue management apparatus 200 includes a sheath 202. The sheath 202 may be generally associated with a catheter body. The sheath 202 may be made from a suitable transparent or translucent material such as fluorinated ethylene propylene (FEP), polytetrafluoroethylene (PTFE), polyphthalamide, polyimide, Nylon, or the like.

The sheath 202 may house an actuation translator 204. The actuation translator 204 enables rotation and translation of an imaging element 206 associated with the actuation translator 204. The actuation translator 204 may actuate the proximal end of the imaging element 206, for example by torque coil, drive shaft, or the like. The actuation translator 204 may actuate the distal end of the imaging element 206, for example by motor, piezoelectric actuator, or the like.

The imaging and tissue management apparatus 200 may include one or more ancillary channels 208. The ancillary channel 208 may be alongside of the sheath 202. The ancillary channel 208 may be made from the same material as the sheath 202 such as fluorinated ethylene propylene (FEP), polytetrafluoroethylene (PTFE), polyphthalamide, polyimide, Nylon, or the like. The sheath 202 and the ancillary channel 208 may be made from a multi-lumen extrusion or a bundle of multiple tubing. The distal exit of the ancillary channel 208 may be at the location covered by the scanning range of an imaging beam 210. Multiple ancillary channels 208 may be included to enable greater coverage of the bodily lumen. In one implementation, the sheath 202 may be steerable via the proximal system to control the position or the orientation of the ancillary channel 208 to tissue 220 associated with the bodily lumen.

The ancillary channel 208 may house a tissue retrieval device 212. The tissue retrieval device 212 may be in the form of a biopsy forceps, a cutter, or the like. The tissue retrieval device 212 will be in a retracted state in the ancillary channel 208 at the time the imaging and tissue management apparatus 200 is guided through the bodily lumen. The ancillary channel 208 enables deployment of the tissue retrieval device 212 at the targeted location. As an example, FIG. 2A illustrates the tissue retrieval device 212 in a retracted state while FIG. 2B illustrates the tissue retrieval device 212 in an extended or deployed state.

The imaging and tissue management apparatus 200 may include one or more balloons 214. The balloons 214 may be inflated via the sheath 202. The balloon 214 may be inflated with air, gas, liquid, or the like. The balloon 214 may be made from a suitable non-compliant, compliant, or semi-compliant material such as polyethylene or other polyolefins, polyurethane, flexible polyvinylchloride, Nylon, or the like. An exterior surface of the balloon 214 may be smooth or substantially smooth. Alternatively, the exterior surface of the balloon 214 may be textured with protuberances, or the like, to aid in anchoring the imaging and tissue management apparatus 200 to tissue 220 associated with the bodily lumen.

In addition to the ancillary channel 208, the imaging and tissue management apparatus 200 may include a distal container 216 located distal of the imaging and tissue management apparatus 200. The distal container may be a smooth shape. For example, the distal container 216 may include a cylinder with a hemispherical end. In general, the distal container 216 may be made of a polymer, such as polyamides, polyurethanes, Nylon, polyethylenes, polyether block amide, polyester, polycarbonate, polypropylene, or the like. The distal container 216 may be used to collect or store the tissue samples removed by the tissue retrieval tool 212, without the need to retract the tissue retrieval tool 212 into the ancillary channel 208. As an example, FIG. 2B shows a biopsy sample 222 of tissue 220 removed by the tissue retrieval tool 212 and retained by the distal container 216.

The imaging element 206 may be coupled to a fiber optic line. The fiber optic line may be contained or housed within the sheath 202. The fiber optic line may be coupled to a portion of the imaging and tissue management apparatus 200 that enables OCT and/or OFDI methods and technologies. The imaging element 206 is functional for circumferential scanning by way of at least the rotation of actuation translator 204. Helical scanning can also be accomplished by simultaneous rotation and pull back by the actuation translator 204.

The imaging element 206 is capable of manipulating, directing, and/or focusing the imaging beam 210 on the tissue 220 during deployment of the tissue retrieval device 212. Light reflected from the tissue 220 may be processed by the imaging element 206 and conveyed to data processing systems associated with the imaging and tissue management apparatus 200 via the fiber optic line, or the like. The processed tissue information enables the guidance of the tissue retrieval process, such as direct visualization of the tissue retrieval tool 212 or the tissue 220 removed by the tissue retrieval tool 212. In one implementation, the reflected light is conveyed wirelessly to the data processing systems associated with the imaging and tissue management apparatus 200.

The sheath 202 may include one or more registration markers 218. The registration marker 218 may be associated with the sheath 202 in the portion covered by the scanning range of the imaging beam 210. The registration marker 218 may provide contrast for OCT/OFDI and at least one other imaging modality to enable positional registration, such as white light endoscopy, ultrasound, fluoroscopy, or the like. In one implementation, the registration marker 218 is made from radiopaque materials for visualization in fluoroscopy, such as barium sulfate, bismuth compounds, tungsten, or the like.

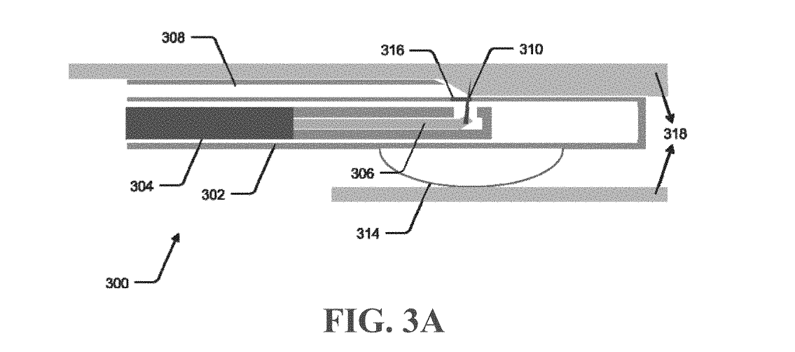

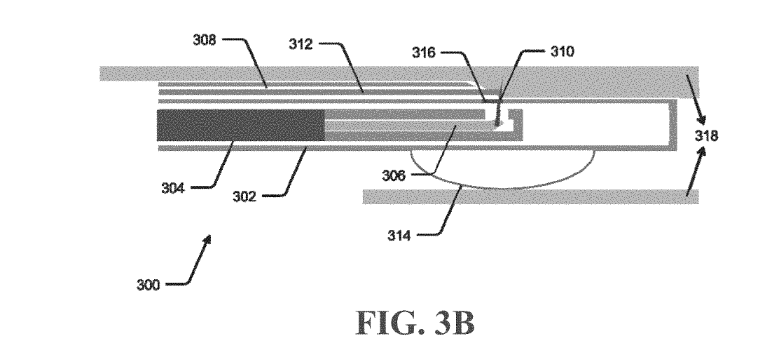

FIGS. 3A and 3B illustrate a third exemplary imaging and tissue management apparatus 300. The third exemplary imaging and tissue management apparatus 300 may implement optical coherence tomography (OCT) and/or optical frequency domain imaging (OFDI) tissue imaging methods and technologies. The third exemplary imaging and tissue management apparatus 300 may alternatively implement other scanning optical imaging modalities, such as fluorescence, spectroscopy, or the like. Furthermore, the imaging and tissue management apparatus 300 may implement other tissue imaging methods and technologies. In one implementation, the third exemplary imaging and tissue management apparatus 300 may include a system utilizing at least one of OCT or OFDI modalities. Specifically, the third exemplary imaging and tissue management apparatus 300 may be capable of detecting electromagnetic radiation, such as a back reflected light, from one or more portions associated with tissue 318. The detected electromagnetic radiation may be processed by the imaging and tissue management apparatus 300 to ascertain information, such as microstructures, associated with the tissue 318.

In one implementation, the third exemplary imaging and tissue management apparatus 300 may include a proximal system including optical fiber, tissue retrieval and treatment functionality, data processing and associated data storage, and the like. As illustrated in FIG. 3, the imaging and tissue management apparatus 300 includes a sheath 302. The sheath 302 may be generally associated with a catheter body. The sheath 302 may be made from a suitable transparent or translucent material such as fluorinated ethylene propylene (FEP), polytetrafluoroethylene (PTFE), polyphthalamide, polyimide, Nylon, or the like.

The sheath 302 may house an actuation translator 304. The actuation translator 304 enables rotation and translation of an imaging element 306 associated with actuation translator 304. The actuation translator 304 may actuate the proximal end of the imaging element 306, for example by torque coil, drive shaft, or the like. The actuation translator 304 may actuate the distal end of the imaging element 306, for example by motor, piezoelectric actuator, or the like.

The imaging and tissue management apparatus 300 may include one or more ancillary channels 308. The ancillary channel 308 may be alongside of the sheath 302. The ancillary channel may be made from the same material as the sheath 302, or a suitable compliant or semi-compliant material such as polyethylene or other polyolefins, polyurethane, flexible polyvinylchloride, Nylon, or the like. The sheath 302 and the ancillary channel may be made from a multi-lumen extrusion or a bundle of multiple tubing. The distal exit of the ancillary channel 308 may be at the location covered by the scanning range of an imaging beam 310. Multiple ancillary channels 308 may be included to enable greater coverage of the bodily lumen. In one implementation, the sheath 302 may be steerable via the proximal system to control the position or the orientation of the ancillary channel 308 to tissue associated with the bodily lumen.

The ancillary channel 308 may allow the introduction of a tissue management device 312. The tissue management device 312 may be in the form of a biopsy forceps, an aspiration needle, an injection needle, an ablation catheter, a coagulation catheter, or the like. The tissue management device 312 may be introduced through the ancillary channel 308 after the imaging and tissue management apparatus 300 reaches the targeted location through the bodily lumen, or in a retracted state in the ancillary channel 308 at the time the imaging and tissue management apparatus 300 is guided through the bodily lumen. The ancillary channel 308 enables deployment of the tissue management device 312 at the targeted location. As an example, FIG. 3A illustrates the ancillary channel without the tissue retrieval device 312 (e.g., with the tissue retrieval device 312 in a retracted state) while FIG. 3B illustrates the tissue retrieval device 312 within the ancillary channel 308 in an extended or deployed state.

The imaging and tissue management apparatus 300 may include one or more balloons 314. The balloons 314 may be inflated via the sheath 302. The balloon 314 may be inflated with air, gas, liquid, or the like. The balloon 314 may be made from a suitable non-compliant, compliant, or semi-compliant material such as polyethylene or other polyolefins, polyurethane, flexible polyvinylchloride, Nylon, or the like. An exterior surface of the balloon 314 may be smooth or substantially smooth. Alternatively, the exterior surface of the balloon 314 may be textured with protuberances, or the like, to aid in anchoring the imaging and tissue management apparatus 300 to tissue 318 associated with the bodily lumen.

The imaging element 306 may be coupled to a fiber optic line. The fiber optic line may be contained or housed within the sheath 302. The fiber optic line may be coupled to a portion of the imaging and tissue management apparatus 300 that enables OCT and/or OFDI methods and technologies. The imaging element 306 is functional for circumferential scanning by way of at least the rotation of actuation translator 304. Helical scanning can also be accomplished by simultaneous rotation and pull back by the actuation translator 304.

The imaging element 306 is capable of manipulating, directing, and/or focusing the imaging beam 310 on the tissue 318 during deployment of the tissue management device 312. Light reflected from the tissue 318 may be processed by the imaging element 306 and conveyed to data processing systems associated with the imaging and tissue management apparatus 300 via the fiber optic line, or the like. The processed tissue information enables the guidance of the tissue management process, such as direct visualization of the tissue management tool 312 or the tissue 318 processed by the tissue management tool 312. In one implementation, the reflected light is conveyed wirelessly to the data processing systems associated with the imaging and tissue management apparatus 300.

The sheath 302 may include one or more registration markers 316. The registration marker 316 may be associated with the sheath 302 in the portion covered by the scanning range of the imaging beam 310. The registration marker 316 may provide contrast for OCT/OFDI and at least one other imaging modality to enable positional registration, such as white light endoscopy, ultrasound, fluoroscopy, or the like. In one implementation, the registration marker 316 is made from radiopaque materials for visualization in fluoroscopy, such as barium sulfate, bismuth compounds, tungsten, or the like.

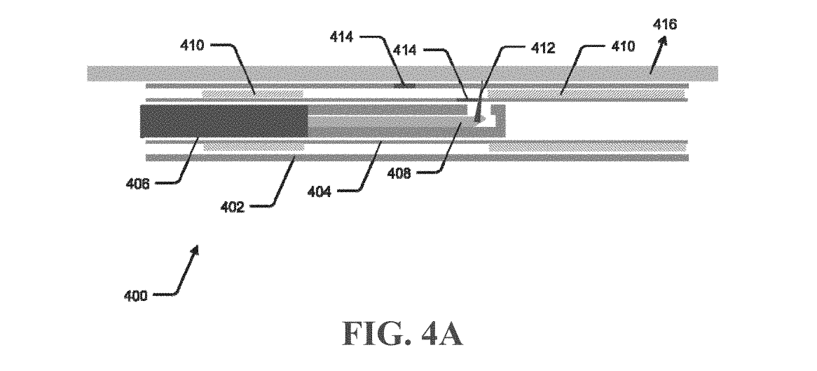

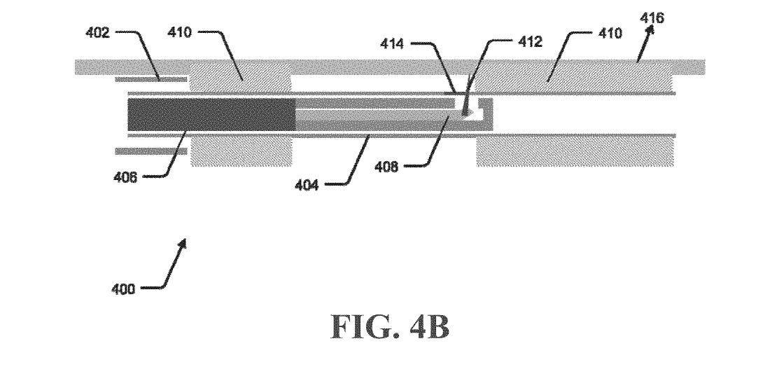

FIGS. 4 and 4B illustrate a fourth exemplary imaging and tissue management apparatus 400. The fourth exemplary imaging and tissue management apparatus 400 may implement optical coherence tomography (OCT) and/or optical frequency domain imaging (OFDI) tissue imaging methods and technologies. The fourth exemplary imaging and tissue management apparatus 400 may alternatively implement other scanning optical imaging modalities, such as fluorescence, spectroscopy, or the like. Furthermore, the imaging and tissue management apparatus 400 may implement other tissue imaging methods and technologies. In one implementation, the fourth exemplary imaging and tissue management apparatus 400 may include a system utilizing at least one of OCT or OFDI modalities. Specifically, the fourth exemplary imaging and tissue management apparatus 400 may be capable of detecting electromagnetic radiation, such as a back reflected light, from one or more portions associated with tissue 416. The detected electromagnetic radiation may be processed by the imaging and tissue management apparatus 400 to ascertain information, such as microstructures, associated with the tissue 416.

In one implementation, the fourth exemplary imaging and tissue management apparatus 400 may include a proximal system including optical fiber, tissue retrieval and treatment functionality, data processing and associated data storage, and the like. As illustrated in FIG. 4, the imaging and tissue management apparatus 400 includes an external sheath 402 and an internal sheath 404. The external sheath 402 may be generally associated with a catheter body and house the internal sheath 404. The external sheath 402 and internal sheath 404 may be made from a suitable material such as fluorinated ethylene propylene (FEP), polytetrafluoroethylene (PTFE), polyphthalamide, polyimide, Nylon, or the like. In one implementation, the external sheath 402 and the internal sheath 404 can be made from a combination of optically clear material in the distal end and kink-resistant material in the remaining portions of the sheaths 402 and 404.

The internal sheath 404 may house an actuation translator 406. The actuation translator 406 enables rotation and translation of an imaging element 408 associated with actuation translator 406. The actuation translator 406 may actuate the proximal end of an imaging element 408, for example by torque coil, drive shaft, or the like. The actuation translator 406 may actuate the distal end of the imaging element 408, for example by motor, piezoelectric actuator, or the like.

The imaging and tissue management apparatus 400 may include one or more cytology brushes 410. The cytology brush 410 may include one or more individual bristles or sponges on the distal circumference of the internal sheath 404, excluding the location covered by the scanning range of an imaging beam 412. The cytology brush 410 may be made from suitable flexible material such as Nylon, stainless steel, or the like. In one implementation, the external sheath 402 may be steerable via the proximal system to operate the cytology brush 410 to retrieve tissue 416 associated with the bodily lumen.

The internal sheath 404 and the cytology brush 410 may be fully retracted inside of the external sheath 402 at the time the imaging and tissue management apparatus 400 is guided through the bodily lumen. The external sheath 402 enables deployment of the internal sheath 404 and the cytology brush 410 at the targeted location. As an example, FIG. 4A illustrates the cytology brush 410 in a retracted state while FIG. 4B illustrates the cytology brush 410 in an extended or deployed state.

The imaging element 408 may be coupled to a fiber optic line. The fiber optic line may be contained or housed within the internal sheath 404. The fiber optic line may be coupled to a portion of the imaging and tissue management apparatus 400 that enables OCT and/or OFDI methods and technologies. The imaging element 408 is functional for circumferential scanning by way of at least the rotation of actuation translator 406. Helical scanning can also be accomplished by simultaneous rotation and pull back by the actuation translator 406.

The imaging element 408 is capable of manipulating, directing, and/or focusing the imaging beam 412 on the tissue 416 during deployment of the internal sheath 404 and the cytology brush 410. Light reflected from the tissue 416 may be processed by the imaging element 406 and conveyed to data processing systems associated with the imaging and tissue management apparatus 400 via the fiber optic line, or the like. The processed tissue reflection information enables the guidance of the tissue brushing process. In one implementation, the reflected light is conveyed wirelessly to the data processing systems associated with the imaging and tissue management apparatus 400.

The external sheath 402 and the internal sheath 404 may include one or more registration markers 414. The registration markers 414 may be associated with the external sheath 402 and/or the internal sheath 404 in the portion covered by the scanning range of the imaging beam 412. The registration markers 414 may provide contrast for OCT/OFDI and at least one other imaging modality to enable positional registration, such as white light endoscopy, ultrasound, fluoroscopy, or the like. In one implementation, the registration markers 414 are made from radiopaque materials for visualization in fluoroscopy, such as barium sulfate, bismuth compounds, tungsten, or the like.

The embodiments have been described and illustrated as including various structures, elements, and operational functionalities. Those described various structures, elements, and operational functionalities may apply to and be used with each of the embodiments described herein.

Furthermore, while imaging apparatuses with tissue removal channels have been described with reference to certain embodiments, it will be understood by those skilled in the art that various changes may be made and equivalents may be substituted without departing from the spirit and scope of the claims of the application. Other modifications may be made to adapt a particular situation or material to the teachings disclosed above without departing from the scope of the claims. Therefore, the claims should not be construed as being limited to any one of the particular embodiments disclosed, but to any embodiments that fall within the scope of the claims.

1. An imaging apparatus, comprising:
a sheath;
an imaging element disposed in the sheath;
an ancillary channel associated with the sheath; and
a tissue management device disposed in the ancillary channel, the tissue management device configured to be deployable and retractable via the ancillary channel.

2. The imaging apparatus according to claim 1, wherein the ancillary channel is alongside of the sheath.

3. The imaging apparatus according to claim 1, wherein the imaging element is disposed in a portion of the sheath associated with a distal exit of the ancillary channel.

4. The imaging apparatus according to claim 1, wherein the tissue management device is configured to be introducible and removable via the ancillary channel.

5. The imaging apparatus according to claim 1, further comprising an actuation translator coupled to the imaging element, the actuation translator configured to rotate and translate the imaging element.

6. The imaging apparatus according to claim 1, further comprising a motor, the motor configured to rotate the imaging element.

7. The imaging apparatus according to claim 1, further comprising an inflatable balloon associated with the sheath, the inflatable balloon configured to be inflatable via the sheath.

8. The imaging apparatus according to claim 1, further comprising a container associated with a distal end of the sheath, the tissue management device configured to be deployed via an exit of the ancillary channel into the container.

9. The imaging apparatus according to claim 1, further comprising one or more registration markers associated with the sheath, the one or more registration markers configured to provide contrast to two or more imaging modalities.

10. An imaging apparatus, comprising:
an external sheath;
an internal sheath disposed within the external sheath;
an imaging element disposed in the internal sheath; and
a cytology brush associated with the internal sheath.

11. The imaging apparatus according to claim 10, wherein the cytology brush covers a distal circumference of the internal sheath.

12. The imaging apparatus according to claim 10, wherein a portion of the internal sheath associated with the cytology brush is configured to be deployable and retractable via the external sheath.

13. The imaging apparatus according to claim 10, wherein the imaging element is disposed in a portion of the internal sheath that does not include the cytology brush.

14. The imaging apparatus according to claim 10, further comprising an actuation translator coupled to the imaging element, the actuation translator configured to rotate and translate the imaging element.

15. The imaging apparatus according to claim 10, further comprising a motor, the motor configured to rotate the imaging element.

16. The imaging apparatus according to claim 10, further comprising one or more registration markers associated with at least one of the external sheath and the internal sheath, the one or more registration markers configured to provide contrast to two or more imaging modalities.

17. A method for retrieving a tissue sample with an imaging and tissue management device, comprising:
detecting back reflected light from tissue within a bodily lumen;
guiding an ancillary channel of the imaging and tissue management device through the bodily lumen based on the detected back reflected light; and
deploying a tissue retrieval device housed in the ancillary channel to obtain the tissue sample.

18. The method of claim 17, further comprising retracting the tissue retrieval device into the ancillary channel when guiding the ancillary channel of the imaging and tissue management device through the bodily lumen.

19. The method of claim 17, further comprising placing the tissue sample into a distal container of the imaging and tissue management device.

20. The method of claim 17, further comprising inflating a balloon to anchor the imaging and tissue management device to the tissue within the bodily lumen.
